(19) United States
(12) Patent Application Publication
(10) Pub. No.: US 2022/0044792 A1
(43) Pub. Date: Feb. 10, 2022

(54) SYSTEM AND METHOD TO PROVIDE TAILORED EDUCATIONAL SUPPORT BASED ON DEVICE USAGE IN A HEALTHCARE SETTING

(71) Applicant: KONINKLIJKE PHILIPS N.V., EINDHOVEN (NL)

(72) Inventors: Paul Anthony SHRUBSOLE, ARNHEM (NL); Doortje VAN DE WOUW, EINDHOVEN (NL); Victor Paulus Marcellus VLOEMANS, SON EN BREUGEL (NL)

(21) Appl. No.: 17/393,480

(22) Filed: Aug. 4, 2021

Related U.S. Application Data
(60) Provisional application No. 63/063,456, filed on Aug. 10, 2020.

(51) Int. Cl. G16H 30/40

(52) U.S. Cl. CPC G16H 30/40

(57) ABSTRACT

A method (100) of providing education content to medical professionals includes: extracting inefficiencies or errors from imaging examination records of a medical facility; receiving context information related to the medical facility; selecting one or more content units (32) to be delivered to a medical professional of the medical facility based on the inefficiencies or errors and the context information; and delivering the selected one or more content units to a mobile device or workstation (18) operable by the medical professional.

This application claims the benefit of U.S. Provisional Application No. 63/063,456, filed on 10 Aug. 2020. This application is hereby incorporated by reference herein.

### FIELD

The following relates generally to the personalized education arts, data-driven education arts, education delivery strategy arts, adaptive education arts, and related arts.

### BACKGROUND

In-hospital medical devices (such as magnetic resonance (MR) scanners, image guided therapy (IGT) systems, and so forth) are complex systems that require significant amounts of training and expertise to be used effectively by hospital staff. This makes it difficult to keep all of the relevant users (e.g., technologists, nurses, radiologists, cardiologists etc.) up to date with new features and protocols that are made available for such devices. To resolve this, traditional educational courses, webinars, apps, technical support, simulators, etc., are delivered to the relevant users. However, these traditionally educational contexts may not match the context of a professional team of users needing education in the context of the product's usage. The educational content may include materials that are distantly related, or unrelated, to the product's usage by a particular user or group of users; and conversely may omit, or provide limited coverage of, materials that would be particularly helpful to those user(s).

Thus, these existing approaches tend to be unsatisfactory and inflexible with regard to many factors: staff turn-around, hospital context not taken into account, too generic, too reactive, limited feedback, and so forth. As the information presented is often general, and not tailored to particular situation or individual (including how an individual might be misusing/underutilizing the product in relation to recommended guidelines or potential optimal results), it is time consuming and not so relevant to the user. Information can be consumed once, but can fade away over time or due to staff turnover. This impacts the effectiveness of hospitals to maximize the potential of the devices and has negative impact on the satisfaction leading to a bad socket retention.

The following discloses certain improvements to overcome these problems and others.

### SUMMARY

In one aspect, a non-transitory computer readable medium stores instructions executable by at least one electronic processor to perform a method of providing education content to medical professionals. The method includes: extracting inefficiencies or errors from imaging examination records of a medical facility; receiving context information related to the medical facility; selecting one or more content units to be delivered to a medical professional of the medical facility based on the inefficiencies or errors and the context information; and delivering the selected one or more content units to a mobile device or workstation operable by the medical professional.

In another aspect, an apparatus for providing education content to medical professionals includes: a database storing content units including audio content, video content, multimedia content, recorded simulator sessions, recorded imaging sessions, virtual reality content, interactive content. At least one electronic processor is programmed to: extract inefficiencies or errors from imaging examination records of a medical facility; receive context information related to the medical facility; select one or more of the content units to be delivered to a medical professional of the medical facility based on the inefficiencies or errors and the context information; and deliver the selected one or more content units to a mobile device or workstation operable by the medical professional.

In another aspect, a method of providing education content to medical professionals includes: extracting inefficiencies or errors from imaging examination records of a medical facility; receiving context information related to the medical facility; determining a granularity level of the user, the granularity level being determined from a set of granularity levels, the set of granularity levels including: a hospital level, a department level, a role-based level, an expertise level, and an individual level; selecting one or more content units to be delivered to a medical professional of the medical facility based on the inefficiencies or errors, the context information, and the granularity level; and delivering the selected one or more content units to a mobile device or workstation operable by the medical professional.

One advantage resides in providing situation-specific educational materials to medical professionals.

Another advantage resides in providing educational materials that can be tailored to medical professionals.

Another advantage resides in providing personalized, adaptable, and practice-driven educational materials about medical systems to medical professionals.

Another advantage resides in tailoring the manner of delivery for specific medical professionals or groups of medical professionals.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the disclosure.

FIG. 1 diagrammatically illustrates an illustrative apparatus for providing educational content units in accordance with the present disclosure.

FIG. 2 shows example flowchart operations performed by the apparatus of FIG. 1.

### DETAILED DESCRIPTION

The following discloses a system or infrastructure for delivering targeted educational content to users of medical imaging devices and image guided therapy (IGT) systems. The disclosed systems and methods can target magnetic resonance (MR) systems, but could be more broadly applied to any medical imaging system or medical imaging device, such as a patient monitor, that is computerized and performs automated event logging at sufficiently detailed resolution (e.g., recording timestamped usage information such as device configuration changes, operational setting changes, and so forth), or any medical device that requires training and education to use the medical device effectively in a medical setting.

The disclosed system delivers educational content to radiologists, imaging technologists, doctors, nurses, hospital administrators, and/or other medical personnel in defined modules, and at a targeted level of granularity. For example, there can be five levels of end-user granularity: hospital level; department level; role-based level (e.g. radiologist v technician); expertise/competency level (e.g. senior radiologist v junior radiologist); and individual level. A given implementation may implement only a sub-set of these personalization granularities. For example, in some specific implementations it may not be possible to identify the individual level due to privacy concerns or use of shared (or no) logins.

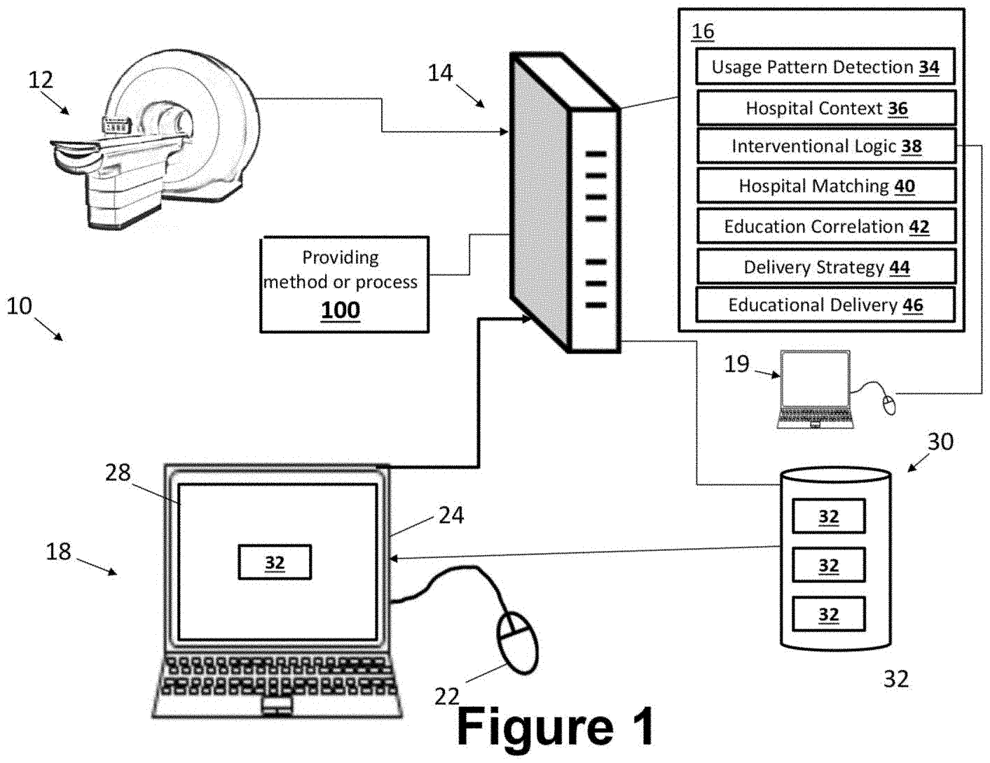

With reference to FIG. 1, an illustrative apparatus 10 for providing education content to medical professionals. The education content can be, for example, about a medical imaging device 12 (or any other suitable medical device). The apparatus 10 includes an electronic processing device 18, such as a workstation computer, cellular telephone, or other mobile device, or more generally a computer. The illustrative workstation 18 may also include a server computer or a plurality of server computers, e.g. interconnected to form a server cluster, cloud computing resource, or so forth, to perform more complex image processing or other complex computational tasks. The workstation 18 includes typical components, such as an electronic processor 20 (e.g., a microprocessor), at least one user input device (e.g., a mouse, a keyboard, a trackball, and/or the like) 22, and a display device 24 (e.g. an LCD display, plasma display, cathode ray tube display, and/or so forth). In some embodiments, the display device 24 can be a separate component from the workstation 18, or may include two or more display devices.

The electronic processor 20 is operatively connected with one or more non-transitory storage media 26. The non-transitory storage media 26 may, by way of non-limiting illustrative example, include one or more of a magnetic disk, RAID, or other magnetic storage medium; a solid state drive, flash drive, electronically erasable read-only memory (EEROM) or other electronic memory; an optical disk or other optical storage; various combinations thereof; or so forth; and may be for example a network storage, an internal hard drive of the workstation 18, various combinations thereof, or so forth. It is to be understood that any reference to a non-transitory medium or media 26 herein is to be broadly construed as encompassing a single medium or multiple media of the same or different types. Likewise, the electronic processor 20 may be embodied as a single electronic processor or as two or more electronic processors. The non-transitory storage media 26 stores instructions executable by the at least one electronic processor 20. The instructions include instructions to generate a visualization of a graphical user interface (GUI) 28 for display on the display device 24.

The workstation 18 (or cellphone or other mobile device used by a medical professional to consume educational materials delivered by the system) is also in communication with a server computer 14, which can include one or more databases 30 that stores the educational content (or can constitute a non-transitory computer readable medium itself). The educational content can be organized into semantically described educational content units 32, which can be organized hierarchically (or, in the case of natural language processing (NLP) based topic extraction, can be organized as a knowledge graph). A content manager (not shown) can be included for organizing and tagging the content units 32. The educational content units 32 may include audio, video, text, and/or multimedia content (e.g., virtual reality presentations). The content may also include simulator sessions (which can be recorded), recorded actual imaging sessions, virtual reality (VR) content, Augmented reality (AR) content, interactive content such as quizzes, and/or so forth. The content units 32 of the database 30 may be generated manually or semi-automatically and may be combined for consumption in sequence (e.g. with automated semantical tagging based on natural language processing of the content). The content units 32 may also be parameterized, such that different values of the content parameters may adjust the portion(s) and/or way the content is delivered. For example, a content unit 32 for educating an imaging technician who operates a magnetic resonance (MR) scanner about use of C-SENSE compression during MR imaging may have a parameter for whether the usage situation requires high speed or high image quality, and the content may then be tailored on that basis (high speed resulting in a recommendation of high C-SENSE compression, versus high image quality resulting in a recommendation of low C-SENSE compression). The content units 32 may also be generated by auto-configuring playback parameters of a simulator representing the used device based on input patterns on prior usage and playing out the simulation to the user. The workstation 18 is configured to receive one or more content units 32 from the database 30 that are recommended for the user, and output the retrieved content units (e.g., on the display device 24).

The server computer 14 is configured to recommend one or more of the content units 32. To do so, the server computer 14 is programmed to implement one or more modules programmed onto at least one electronic processor 16 thereof. A usage pattern detection module 34 in the context of medical imaging device usage education is suitably a data mining module configured to receive imaging examination records from the medical imaging device 12 or other medical device. The imaging examination records can include, for example, machine logs files, records of patient safety alerts, equipment warning alerts, or other alerts generated during imaging examinations, radiology reports, other data generated by imaging devices, call logs of technician calls to remote expert assistance centers, or any other suitable types of data relating to imaging examination processes and operations. From the imaging examination records, the usage pattern detection module 34 is configured to extract usage inefficiencies or errors (either collected over time, or in real-time) at one or more of the five levels of end-user granularities. In some examples, content units 32 can be selected for multiple granularity levels (e.g., a selected content unit can be sent to users of two or more different granularity levels). This approach leverages the observation that MR scanners, as well as computed tomography (CT) scanners, image-guided therapy (IGT) systems, and other medical imaging devices are computerized devices with arrays of sensors providing measurements, actuator operation feedback, and so forth, as well as employing computerized imaging sequences commonly constructed by an imaging technician by retrieving a standard (i.e. template) imaging sequence and adjusting parameters of the retrieved imaging sequence to tailor it for a specific imaging examination. Such computerized medical imaging devices commonly maintain a machine log (or multiple logs or other files) that automatically record operational data such as sensor readings, actuator operation feedback data, executed imaging sequences including parameter settings, and so forth, for example stored as timestamped messages of a log file. The imaging examination records thus provide a source of information for determining usage patterns. The level of granularity at which the usage patterns can be determined (e.g., associated to a specific imaging technician based on login information, or to a radiology work shift based on time stamps) depends upon the workflow protocols employed in a given radiology laboratory or hospital.

On the other hand, typically the available imaging examination records do not provide useful output quality information such as information on image quality (which is an important educational component when learning how to perform imaging-based diagnostics). For example, in many workflow scenarios, any preview images acquired by the medical imaging device prior to collecting the clinical images are discarded, and the final clinical images are usually stored in a separate database, such as a Picture Archiving and Communication System (PACS) database. In these cases, inefficiencies or errors can be detected indirectly, for example as: (i) outliers in the usage pattern of one end-user compared with other end-users (for example, a department or hospital routinely uses a different parameter value or value range compared with other departments or hospitals); and/or (ii) usage pattern of an end-user that differs significantly from an expected usage pattern determined by the vendor.

In one example, an MR scanner feature of C-SENSE compression level (e.g., having a parameter value range 2-32) impacts speed versus image quality. This parameter value is logged, and hence can be tracked amongst end-users. An end user who consistently uses a too-high or too-low value (as indicated by being an outlier and/or differing significantly from the expected value range) is flagged. Another possible basis for flagging usage is if the end user always uses the default C-SENSE compression level, as this may indicate the end user is unaware of the value of adjusting the C-SENSE compression value. More generally, the failure of an end user to utilize an available feature may be flagged as an indication that the user is unaware of the feature or is uncomfortable with utilizing it. In another example, in which an IGT procedure delivers ionizing radiation, the logged events can be used to estimate radiological parameters such as total dose and peak dose, and again an end user who consistently delivers a high or low total or peak dose compared with an expected dose range may be flagged.

In some embodiments, on the other hand, some form of performance feedback may be received that can (also) be utilized for detecting inefficiencies or errors. One way of providing such feedback is to incorporate an end-user rating GUI dialog integrated into an imaging console display (not shown) on the medical imaging device 12 which pops up when the image is presented on the console display (e.g. querying “Is the image satisfactory to you?” and providing “Yes” and “No” selection buttons; or alternatively asking the user to rate image quality on a scale of 1-5). In a more complex embodiment, the console display may be screen-scraped (e.g., using a DVI splitter, screen-sharing software, or the like) to obtain the actual image, which can then be automatically analyzed to quantitatively assess image quality. In such embodiments, the image quality or other feedback information may be leveraged to identify usage inefficiencies or errors. However, such feedback may be limited or precluded due to concerns (e.g., such as HIPAA data privacy in the United States) and/or unwillingness of an end-user institution to introduce additional operations such as image rating dialogs into the imaging workflow.

In other embodiments, an end user, such as an imaging technician, may have access to remote expert assistance. For example, the imaging technician may call a remote expert if the imaging technician is having difficulty with a particular portion of an imaging procedure. The remote expert is based at a remote workstation (e.g. desktop computer, computer terminal or the like, which may be in the same hospital as the imaging technician or in a different location or even in a different state or country. The remote workstation has screen-sharing capability so that the remote expert can see a copy of a display of a controller of the imaging device 12 at the remote workstation. Optionally, the remote workstation may also present a window displaying a video feed received over the Internet from a video camera positioned to provide video of the imaging device, control room, or other key operational location(s). The remote expert and imaging technician communicate via telephone, text messaging, video conferencing (e.g., running in windows on the remote workstation and the imaging device controller or another computer in an imaging bay, respectively), or the like to enable the remote expert to provide advice to the imaging technician regarding the difficult portion of the imaging procedure. Such remote expert assistance can be beneficial as it allows a single senior imaging technician to serve as the remote expert for assisting imaging technicians working in the same or different hospitals (or even in different states or countries), thereby efficiently leveraging the special expertise of the remote expert.

In such embodiments, if the imaging technician calls for assistance from the remote expert for a particular task, such calls can be saved in a remote expert call log that serves as another type of examination record. The usage pattern detection module 34 is programmed to extract such call logs as usage inefficiencies or errors.

A hospital context module 36 is configured to provide information on the hospital context. In some examples, the hospital context module 36 can be an end-user context module or modules, for example also providing individual end-user context information. The hospital context module(s) 36 in some embodiments automatically mines context information from hospital databases. Additionally or alternatively, an end-user user interface (UI) may be implemented via a GUI 28 presented on the display device 24 via which the end-user manually inputs context information. In the latter case, the hospital context module 36 suitably implements an end user interface on the GUI 28 that is accessible at the individual radiologist level, department level, hospital level, or some combination of these, via which the end-user can enter a user profile with information such as content delivery modality preferences or constraints (where the modality may be audio, video, text, simulator, etc.), content delivery time/interval preferences or constraints, content distribution rules, etc.

In some embodiments, other sources of context may be utilized if available, such as real-time location system (RTLS) information used for identifying specific users, or environmental sensors (not shown) used to sense potential issues such as a room light being on during a procedure that is specified to be performed in the dark, or room temperature being too high thereby potentially adversely affecting noise level of positron emission tomography (PET) sensors, and so forth.

An interventional logic module 38 is configured to provide a UI for a user, such as an application specialist, on a workstation 19 operable by the application specialist. This workstation can comprise a back-end of the apparatus 10, with the application specialist being an employee who provides interfacing with end-users of the workstation 18. In one example, the application specialist is employed by the vendor of the medical imaging device and the workstation 19 is at a location controlled by the vendor. In another example, the application specialist is employed by the hospital and the workstation 19 is located in an administrative area of the hospital. Via the interventional logic module 38, the application specialist can adjust context information for specific end-users, handle unaccepted educational unit push operations that are escalated to the application specialist, and so forth. The interventional logic module 38 can include an artificial intelligence (AI) component that, for example, may detect when an end-user has failed to consume an educational unit within an allowable time frame.

A hospital matching module 40 is configured to automatically identify similar hospitals (or, more generally for other levels of end-user granularity, may identify similar departments, similar individuals, et cetera). Optionally, the end-user UI on the GUI 28 may also provide a dialog where an end-user (e.g. hospital) can self-identify similar hospitals.

From the information collected or obtained by the usage pattern detection module 34, the hospital context module 36, the interventional logic module 38, and/or the hospital matching module 40, an education correlation module 42 is configured to automatically select one or more content units 32 to be delivered to end-users on the workstation 18. In some examples, the education correlation module 40 is configured to select one or more content units 32 based on different combinations of error types (e.g., an “error A” and a “error B” can result in selecting a content unit labeled “content C”; while the error A and a different “error D” can result in selecting a content unit labeled “content E”), and so forth). In other examples, the education correlation module 40 is configured to select one or more content units 32 related to training the user on using remote assistance, or training the user on imaging procedures to avoid having to rely on remote assistance, or recommending a user to use remote assistance for particular imaging tasks until training is completed for such tasks. In further examples, the education correlation module 42 is configured to select one or more content units 32 by balancing a frequency of errors detected by the usage pattern detection module 34, or a severity of such errors. For example, a “minor” error that occurs numerous times (e.g., failing to instruct a patient not to move during an image acquisition process) can be rated lower than a more severe error (e.g., incorrectly positioning a patient on the image acquisition device 12 before the image acquisition process begins).

The education correlation module 42 may optionally also employ feedback. For example, the presentation of the education content units 32 on the workstation 18 may include a “like/dislike” question at the end, or some other feedback query dialog, and this information can be used to assess whether the end-user is receiving useful information. The end-user UI on the GUI 28 may also allow for end-users to request content covering a certain topic, and this can be taken into account in selecting the content units 32 to present. Other feedback can be obtained based on whether a particular inefficiency or error continues after the educational content unit 32 covering that issue is presented. If the inefficiency or error continues then the content units 32 can be re-presented to the end-user, and/or the issue may be escalated to the interventional logic module 38 to inform the application specialist that the educational content unit 32 is not resolving the inefficiency or error.

A delivery strategy selection module 44 is configured to automatically select the manner (e.g., times, places, modality (which can include audio and/or text), and so forth) for delivery of the content identified by the educational correlation module. This may be based on end-user profiles indicating preferences and/or constraints on these delivery pathways.

An educational delivery module 46 is configured to handle the actual delivery of the content units 32 to the workstation 18. In some examples, the educational delivery module 46 (where feasible) is configured to determine whether the content was actually consumed. To do so, the educational delivery module 46 can determine whether the user opens the content (e.g. opens a text file) or can be more elaborate by for example monitoring whether an entire video is played. If the content unit 32 has feedback, then this is collected by the educational delivery module 46 and forwarded to the other modules that utilize this feedback. For example, if an end-user fails to consume a content unit 32 within a designated time, this may be escalated to the interventional logic module 38 for review by the application specialist. In some embodiments, the educational delivery module 46 can push urgent content, for example if it is detected that an end-user is about to make a particular error (e.g. setting the radiation level too high in an IGT workflow).

The apparatus 10 is configured as described above to perform an analysis method or process 100 for providing education content units 32 to medical professionals, which can be stored in the database 30 of the server computer 14. The server computer 14 stores instructions which are readable and executable by the at least one electronic processor 16 thereof to perform disclosed operations including performing the method or process 100. In some examples, the method 100 may be performed at least in part by cloud processing.

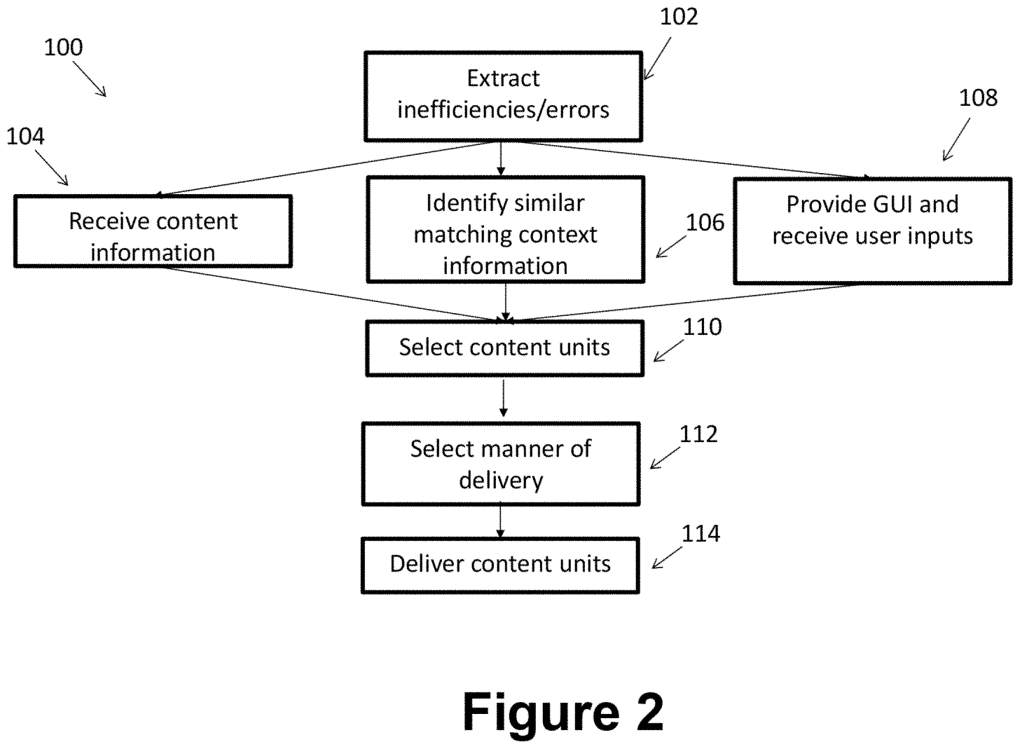

With reference to FIG. 2, and with continuing reference to FIG. 1, an illustrative embodiment of the method 100 is diagrammatically shown as a flowchart. At an operation 102, inefficiencies or errors are extracted from imaging examination records of, for example, a medical facility where the medical imaging device 12 is disposed. The operation 102 can be performed by the usage pattern detection module 34. In some examples, the extracting can include detecting outliers in a usage pattern that differs between users or differs from an expected usage pattern.

At an operation 104, content information related to the medical facility can be received by the server computer 14. The operation 104 can be performed by the hospital context module 36.

At an operation 106, information related to one or more other medical facilities with similar matching context information to the medical facility is identified. The operation 106 can be performed by the hospital matching module 40.

At an operation 108, the GUI 28 is provided on the display device of the workstation 18 with one or more dialogs. The operation 108 can be performed by the interventional logic module 38. The dialogs on the GUI 28 allow the end-user to allow a user to provide one or more user inputs indicating user preferences or constraints on modality of the delivery and/or location of the delivery and/or time of delivery. The dialogs can include, for example, a dialog to edit the context information; a dialog to provide feedback of the one or more content units 32; a dialog to select the one or more other medical facilities; a dialog to request a content unit; a dialog to provide feedback related to an effectiveness or relevance of the selected content units, and so forth.

It will be appreciated that the operations 104-108 can be performed in any suitable order. That is, the operations 104-108 do not necessarily need to be performed in their listed manner. For example, the operation 106 can be performed by the operations 104 and 108; the operation 108 can be performed, followed by the operation 106, and followed by the operation 104; and so forth.

At an operation 110, one or more of the content units 32 are selected to be delivered to a medical professional of the medical facility based on the inefficiencies or errors (i.e., determined at the operation 102) and the context information (i.e., determined at the operations 104-108). The operation 110 can be performed by the education correlation module 42. In some examples, the operation 110 can include determining the granularity level of the user from a set of granularity levels including: a hospital level, a department level, a role-based level, an expertise level, and an individual level. The content units 32 can be selected based on the determined granularity level of the user. In a more particular example, the selected content units 32 can include educational content for the medical imaging device 12.

At an operation 112, a manner for delivery of the selected content units 32 is selected. The operation 112 can be performed by the delivery strategy selection module 44. The selected manner of delivery can include, for example, a delivery device (e.g. the illustrative workstation 18 or a cellphone or other mobile device), time, a place (for example, delivering content units when the medical professional is at a certain location as determined via GPS or other locating service of a cellphone, or an RTLS, or an expected location/time based on a pre-determined clinical workflow), and a modality (e.g., audio, video, text, simulator, etc.) to deliver the selected content units 32. In some examples, the selected manner for delivery can further be based on the user preferences, behaviors, or constraints input to the dialogs of the GUI 28.

At an operation 114, the selected one or more content units 32 are delivered to the workstation 18 operable by the medical professional end-user. The operation 114 can be performed by the educational delivery module 46. In some examples, the operation 114 can include determining (via the educational delivery module 46) whether the delivered content units 32 are completely or otherwise accessed by the end user. If not, a notification (e.g., a visual message or an audio tone) can be sent to the application specialist, who can then update the selected content units 32 accordingly.

The disclosure has been described with reference to the preferred embodiments. Modifications and alterations may occur to others upon reading and understanding the preceding detailed description. It is intended that the exemplary embodiment be construed as including all such modifications and alterations insofar as they come within the scope of the appended claims or the equivalents thereof.

1. A non-transitory computer readable medium storing instructions executable by at least one electronic processor to perform a method of providing education content to medical professionals, the method comprising:
extracting inefficiencies or errors from imaging examination records of a medical facility;
receiving context information related to the medical facility;
selecting one or more content units to be delivered to a medical professional of the medical facility based on the inefficiencies or errors and the context information; and
delivering the selected one or more content units to a mobile device or workstation operable by the medical professional.

2. The non-transitory computer readable medium of claim 1, wherein the method further includes:
providing, on a display device of a workstation, a user interface (UI) with one or more dialogs to allow a user to provide one or more user inputs indicating user preferences or constraints on modality of the delivery and/or location of the delivery and/or time of delivery; and
selecting a manner for the delivery of the selected one or more content units based on the user preferences or constraints.

3. The non-transitory computer readable medium of claim 1, wherein the method further includes:
identifying information related to one or more other medical facilities with similar matching context information to the medical facility.

4. The non-transitory computer readable medium of claim 2, wherein the one or more dialogs include one or more of: a dialog to edit the context information; a dialog to provide feedback of the selected one or more content units; a dialog to select the one or more other medical facilities; a dialog to request a content unit; a dialog to provide feedback related to an effectiveness or relevance of the selected content units.

5. The non-transitory computer readable medium of claim 1, wherein the delivering includes:
determining whether the delivered one or more content units were completed or accessed; and
in response to the one or more content units not being completed or accessed, generating a notification to a workstation operable by a user different from the medical professional.

6. The non-transitory computer readable medium of claim 1, wherein the method further includes:
selecting a manner for delivery of the selected one or more content units.

7. The non-transitory computer readable of claim 6, wherein the selected manner includes one or more of a time, a place, and a modality to deliver the selected content units.

8. The non-transitory computer readable of claim 1, wherein the extracting includes detecting outliers in a usage pattern that differs between users or differs from an expected usage pattern.

9. The non-transitory computer readable medium of claim 1, wherein the selecting includes:
determining a granularity level of the user; and
wherein the selecting of the one or more content units is further based on the determined granularity level.

10. The non-transitory computer readable medium of claim 9, wherein the granularity level is determined from a set of granularity levels, the set of granularity levels including: a hospital level, a department level, a role-based level, an expertise level, and an individual level.

11. The non-transitory computer readable medium of claim 10, wherein the selecting includes:
determining a granularity level of the user; and
wherein the selecting of the one or more content units further includes selecting one or more content units for a plurality of determined granularity levels.

12. The non-transitory computer readable medium of claim 1, wherein the content units are stored in a database, the content units being selected from a group consisting of audio content, video content, multimedia content, recorded simulator sessions, recorded imaging sessions, virtual reality content, mixed/augmented reality content, interactive content.

13. The non-transitory computer readable medium of claim 1, wherein the method provides education content to medical imaging professionals operating medical imaging devices, and wherein:
the inefficiencies or errors are extracted from one or more imaging examination records of one or more of the medical imaging devices; and
the selected one or more content units pertain to operation of at least one of the medical imaging devices.

14. The non-transitory computer readable medium of claim 1, wherein extracting inefficiencies or errors from imaging examination records of a medical facility includes:
extracting inefficiencies or errors from imaging examination records related to remote assistance of a user from a remote expert; and selecting of one or more content units includes:
selecting one or more content units related to the remote assistance.

15. The non-transitory computer readable medium of claim 1, wherein selecting of one or more content units includes:
selecting one or more content units based on combinations of inefficiencies or error types.

16. The non-transitory computer readable medium of claim 1, wherein selecting of one or more content units includes:
selecting one or more content units based on a frequency and/or a severity of the extracted inefficiencies or error types.

17. An apparatus for providing education content to medical professionals, the apparatus comprising:
a database storing content units, the content units being selected from a group consisting of audio content, video content, multimedia content, recorded simulator sessions, recorded imaging sessions, virtual reality content, interactive content; and
at least one electronic processor programmed to:
extract inefficiencies or errors from imaging examination records of a medical facility;
receive context information related to the medical facility;
select one or more of the content units to be delivered to a medical professional of the medical facility based on the inefficiencies or errors and the context information; and
deliver the selected one or more content units to a mobile device or workstation operable by the medical professional.

18. The apparatus of claim 17, wherein the at least one electronic processor is further programmed to:
provide, on a display device of the workstation, a user interface (UI) with one or more dialogs to allow a user to provide one or more user inputs indicating user preferences or constraints on modality of the delivery and/or location of the delivery and/or time of delivery; and
select a manner for the delivery of the selected one or more content units based on the user preferences or constraints.

19. The apparatus of claim 17, wherein the at least one electronic processor is further programmed to:
identify information related to one or more other medical facilities with similar matching context information to the medical facility.

20. The apparatus of claim 18, wherein the one or more dialogs include one or more of: a dialog to edit the context information; a dialog to provide feedback of the selected one or more content units; a dialog to select the one or more other medical facilities; a dialog to request a content unit; a dialog to provide feedback related to an effectiveness or relevance of the selected content units.

21. The apparatus of claim 17 wherein the at least one electronic processor is further programmed to:
select a manner for delivery of the selected one or more content units.

22. The apparatus of claim 17, wherein the at least one electronic processor is further programmed to:
determine a granularity level of the user, the granularity level being determined from a set of granularity levels, the set of granularity levels including: a hospital level, a department level, a role-based level, an expertise level, and an individual level; and
wherein the selecting of the one or more content units is further based on the determined granularity level.

23. The apparatus of claim 17, wherein the at least one electronic processor is further programmed to:
extract the inefficiencies or errors from one or more imaging examination records of one or more of the medical imaging devices; and
select one or more content units pertaining to operation of at least one of the medical imaging devices.

24. A method of providing education content to medical professionals, the method comprising:
extracting inefficiencies or errors from imaging examination records of a medical facility;
receiving context information related to the medical facility;
determining a granularity level of the user, the granularity level being determined from a set of granularity levels, the set of granularity levels including: a hospital level, a department level, a role-based level, an expertise level, and an individual level;
selecting one or more content units to be delivered to a medical professional of the medical facility based on the inefficiencies or errors, the context information, and the granularity level; and
delivering the selected one or more content units to a mobile device or workstation operable by the medical professional.
